(19) United States
(12) Patent Application Publication
(10) Pub. No.: US 2004/0018632 A1
(43) Pub. Date: Jan. 29, 2004

(54) Hydrogen processing unit for fuel cell storage systems

(76) Inventors: Mohsen D. Shabana, Ann Arbor, MI (US); Adrian B. Chernoff, Royal Oak, MI (US)

Correspondence Address: KATHRYN A MARRA, General Motors Corporation, Mail Code 482-C23-B21, P.O. Box 300, Detroit, MI, 48265-3000, US

(21) Appl. No.: 10/202,463

(22) Filed: Jul. 24, 2002

(51) Int. Cl. G01N 33/00

(52) U.S. Cl. 436/144; 436/181

(57) ABSTRACT

A hydrogen processing unit is provided for attachment between a fuel cell stack (or stacks) and a hydrogen storage media (or a plurality of hydrogen storage media). The hydrogen processing unit includes a heat exchanger, a hydrogen filter, a hydrogen pressure regulator, and a hydrogen compressor to enable selective attachment of hydrogen storage media in different forms, including compressed gas, liquid or solid hydride. Alternatively, the hydrogen processing unit may include a catalyst to enable attachment of hydrogen storage media in the form of chemical hydrides.

### TECHNICAL FIELD

The present invention relates to a hydrogen processing unit configured to receive hydrogen stored in different forms and to deliver hydrogen gas to the fuel cell on a fuel cell powered vehicle.

### BACKGROUND OF THE INVENTION

Recently, fuel cells have been discussed as a source of propulsion for vehicles to replace the internal combustion engine. The fuel cell is a device which converts hydrogen and oxygen into electricity. It achieves this using a process which is the reverse of the electrolysis of water. It is particularly desirable for use in vehicles because its only by products are air and water.

Fuel cell systems for vehicles typically include an air compressor module, a humidifier module, a fuel cell module, and an inverter module. In the air compressor module, ambient air is filtered and compressed to the fuel cell operating pressure. This module also recovers water generated from the fuel cell stack and recirculates it to the humidifier module. In the humidifier module, hydrogen fuel and compressed air are humidified by water from a de-ionized water cooling circuit. Hydrogen and air are then delivered to the fuel cell stack. In the fuel cell stack, electricity is generated to power the vehicle. As an example, the electrolyte in the fuel cell stack may be potassium hydroxide. This chemical is controlled at 70° C. during full power operating conditions. The liquid potassium hydroxide is positioned between porous membranes (catalysts), which are disposed between a mesh anode and cathode, through which hydrogen and oxygen are injected, respectively. Ion recombination takes place at the porous membranes. As a result of this process the electrons taken from the hydrogen (i.e. electricity) travel to the inverter module which includes motor inverters to drive the wheels.

Typically, fuel cell systems include a hydrogen source which stores the hydrogen in a selected state or phase, such as compressed gas, liquid or solid hydride.

### SUMMARY OF THE INVENTION

The present invention provides a hydrogen processing unit positioned between hydrogen storage media and a fuel cell stack (or stacks) for use in a fuel cell vehicle. The hydrogen processing unit is a standardized unit which is configured to receive and process hydrogen from different types of storage media which store the hydrogen in a selected state or phase, such as compressed gas, liquid or solid hydride, and to assure that the fuel cell stack receives hydrogen in the same form, and at the same pressure, temperature, humidity and purity, regardless of the type of hydrogen storage media. Accordingly, the hydrogen processing unit may be installed on a fuel cell powered vehicle using any of the different types of hydrogen storage media (i.e. compressed gas, liquid or solid hydride). Further, on a particular fuel cell powered vehicle, a hydrogen processing unit would enable different types of hydrogen storage media to be interchanged on the vehicle. For example, when improvements are made to hydrogen storage media over the lifetime of the vehicle having the hydrogen processing unit thereon, the hydrogen processing unit would be configured such that the particular hydrogen storage media used on that vehicle may be replaced with a different type of media (i.e. compressed gas media could be replaced with liquid or solid hydrogen storage, etc.).

Accordingly, the invention provides a hydrogen processing unit for a fuel cell powered vehicle. The hydrogen processing unit includes a heat exchanger, a hydrogen filter, a hydrogen pressure regulator, and a hydrogen compressor to enable selective attachment of any of three different types of hydrogen storage media, including compressed gas, liquid, and solid hydride.

If the storage media is compressed gas, hydrogen directed to the hydrogen processing unit would enter a hydrogen pressure regulator (a throttle valve), which would drop the hydrogen pressure to the fuel cell stack pressure (or to a desired pressure). Also, a heat exchanger in the hydrogen processing unit would heat the gaseous hydrogen to the desired cell stack temperature.

If the hydrogen storage media is liquid, a heater inside the storage media would evaporate the liquid to a gas form by using waste heat or an electric heater. This low pressure hydrogen gas would then go to the hydrogen processing unit, wherein a compressor would compress the gas to the fuel cell stack pressure (or to a desired pressure), and a heat exchanger would heat the gas to a desired stack temperature.

If the hydrogen storage media is a solid media, such as a solid hydride, an electrical coil or other type heater inside of the hydrogen storage media would heat the storage media so that the hydrogen gas is expelled. The hydrogen gas would then be delivered to the hydrogen processing unit, where a heat exchanger heats the gas to the stack temperature, a hydrogen filter, such as a chemical filter (catalyst) or mechanical filter, filters the hydrogen to a desired level of purity, and a hydrogen compressor in the hydrogen processing unit raises the pressure to the fuel cell stack pressure (or to a desired pressure).

As an alternative, the hydrogen processing unit may include a catalyst for processing hydrogen storage in the form of chemical hydrides which generate hydrogen when mixed with water in the presence (or absence) of a catalyst.

The above objects, features, advantages and other objects, features and advantages of the present invention are readily apparent from the following detailed description of the best modes for carrying out the invention when taken in connection with the claims.

### BRIEF DESCRIPTION OF THE DRAWINGS

FIG. 1 is a schematic illustration of a fuel cell system incorporating a hydrogen processing unit which is selectively connectable to a variety of different hydrogen storage media in accordance with the present invention; and

FIG. 2 is a schematic illustration of three compressed gas hydrogen storage containers connected to a hydrogen processing unit through a common manifold for delivery to fuel cell stacks in a vehicle fuel cell system in accordance with the invention.

### DESCRIPTION OF THE PREFERRED EMBODIMENTS

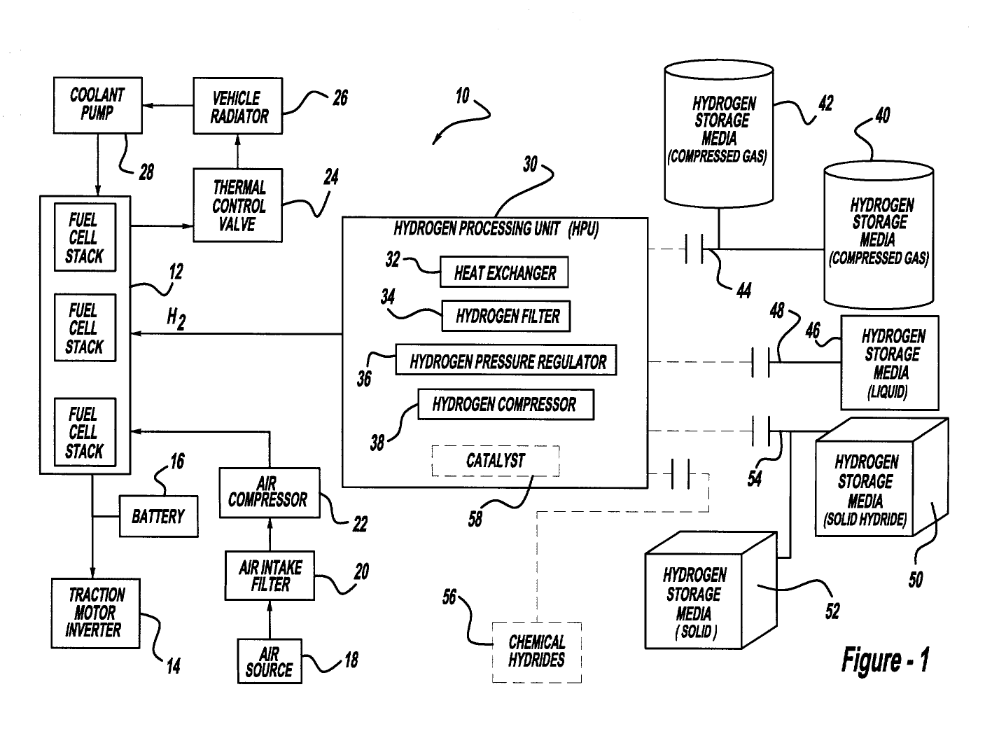

Referring to FIG. 1, a fuel cell system 10 is shown for use in a vehicle in accordance with the present invention. The fuel cell system 10 includes a plurality of fuel cell stacks 12. The fuel cell stacks 12 may be, for example, proton exchange membrane or polymer electrolyte membrane (PEM) fuel cells which convert hydrogen and oxygen into electricity. The electricity is conveyed to the traction motor inverter 14 to drive the vehicle. The electricity may also charge a battery 16, which then powers the traction motor inverter 14 to power the vehicle.

An air compressor module includes a source of ambient air 18 which feeds air through an intake filter 20 to an air compressor 22. The air compressor 22 compresses the air to a desired fuel cell stack pressure.

A proton exchange membrane fuel cell typically operates in the range of 70°-85° C. A heat exchanger module is provided to maintain the fuel cell stacks at the desired operating temperature. The heat exchanger module includes a thermal control valve 24, which leads to the vehicle radiator 26 where heat is dissipated, and the coolant pump 28 pumps the cooled coolant (water) back to the fuel cell stack 12 in a controlled manner.

Hydrogen for the fuel cell stacks is delivered from the hydrogen processing unit 30. In the preferred embodiment, the hydrogen processing unit 30 includes a heat exchanger 32, a hydrogen filter 34, a hydrogen pressure regulator 36, and a hydrogen compressor 38. Accordingly, the hydrogen processing unit is configured to receive and process hydrogen from a variety of different hydrogen storage media, including compressed gas, liquid, and solid hydride media. Therefore, the hydrogen processing unit may be connected to a compressed gas, liquid, or solid hydride hydrogen storage container, or containers, and deliver hydrogen gas to the fuel cell stacks 12 at a desired pressure, temperature, humidity and purity. The different types of storage media may be interchangeably connected to the hydrogen processing unit.

By way of example, if the hydrogen storage media is compressed gas, a common manifold 44 would carry the hydrogen gas from the storage tanks 40, 42 to the hydrogen processing unit 30 wherein a hydrogen pressure regulator 36, such as a throttle valve, would drop the pressure to a desired stack pressure, such as 10 bars for example to provide 1.5 to 3 bars at the fuel cell stacks. Also, the heat exchanger 32 in the hydrogen processing unit 30 would heat the gas to a desired stack temperature, such as 80° C. The compressed gas would be stored in the storage tanks 40, 42 at 5,000-10,000 p.s.i.

Alternatively, the hydrogen may be stored as a liquid, such as in the storage tank 46. The liquid hydrogen is super-cooled and stored cryogenically. An electric heater or waste heat removal from the fuel cell stacks may be used to heat the super-cooled liquid hydrogen to evaporate the liquid to generate hydrogen gas. The hydrogen gas is fed to the hydrogen processing unit through the manifold 48. In the hydrogen processing unit, the hydrogen compressor 38 compresses the hydrogen gas to the desired pressure (1 to 10 bars), and the heat exchanger 32 heats the gas to the desired stack temperature (80° C.) for delivery to the fuel cell stacks 12.

The hydrogen processing unit 30 is also configured to receive and process hydrogen stored in the solid form, such as in the storage media 50, 52. The solid hydrogen storage media is in the form of a metal hydride alloy. Metal hydrides are formed when metal atoms bond with hydrogen to form stable compounds. The alloys absorb hydrogen gas, and store the gas at low pressures, such as 6-10 atmospheres. Within the storage media 50, 52, an electric coil may be provided to heat a portion of the metal hydride alloy to cause discharge of the hydrogen gas through the manifold 54 to the hydrogen processing unit 30. In the hydrogen processing unit 30, the heat exchanger 32 heats the gas to the desired fuel stack operating temperature, and the hydrogen filter 34, such as a chemical filter (catalyst) or mechanical filter, filters the hydrogen gas to the desired purity. The filter removes any entrained vapors, reactants, salt, effluent or other impurities. Accordingly, the hydrogen gas is dried (0% humidity) and purified by the filter 34. The compressor 38 may also compress the gas if necessary. The hydrogen gas then is passed from the hydrogen processing unit 30 to the fuel cell stack 12.

Another form of solid hydrogen storage is sodium hydride (sodium tetrahydridoborate) represented by storage media 52 of FIG. 1. Sodium hydride is a powder which, when dissolved in water or an alkaline solution, in the presence of a platinum catalyst (which is the anode), releases hydrogen, and hydrogen is also released from the water. Another form of solid hydrogen storage is sodium borohydride, represented in FIG. 1 as storage media 52. Sodium borohydride is a salt which produces hydrogen when dissolved in water in the presence of a catalyst.

Some storage options for alternative solid hydrogen forms include plastic encapsulated hydride pellets, wherein hydrogen is produced when the pellets are cut and immersed in water, as well as carbon nanotubes, carbon fullerenes, and glass microspheres.

As further illustrated in FIG. 1, chemical hydrides 56, such as sodium borohydride or potassium borohydride, may require a catalyst 58, which may be optionally provided in the hydrogen processing unit 30 to generate hydrogen when the chemical hydrides are mixed with water in the presence of the catalyst 56.

Accordingly, the hydrogen processing unit 30 is configured to selectively receive hydrogen gas from a compressed gas source, a liquid hydrogen source, or a solid hydrogen source. Therefore, for example, the hydrogen processing unit 30 may installed as part of a fuel cell system 10 in a vehicle. As developments occur in the design, manufacture and use of different forms of hydrogen storage media, the originally installed hydrogen storage media may be removed from the vehicle and replaced by a different type of hydrogen storage media which includes hydrogen stored in a different state (gas, liquid or solid), without modification of the hydrogen processing unit 30.

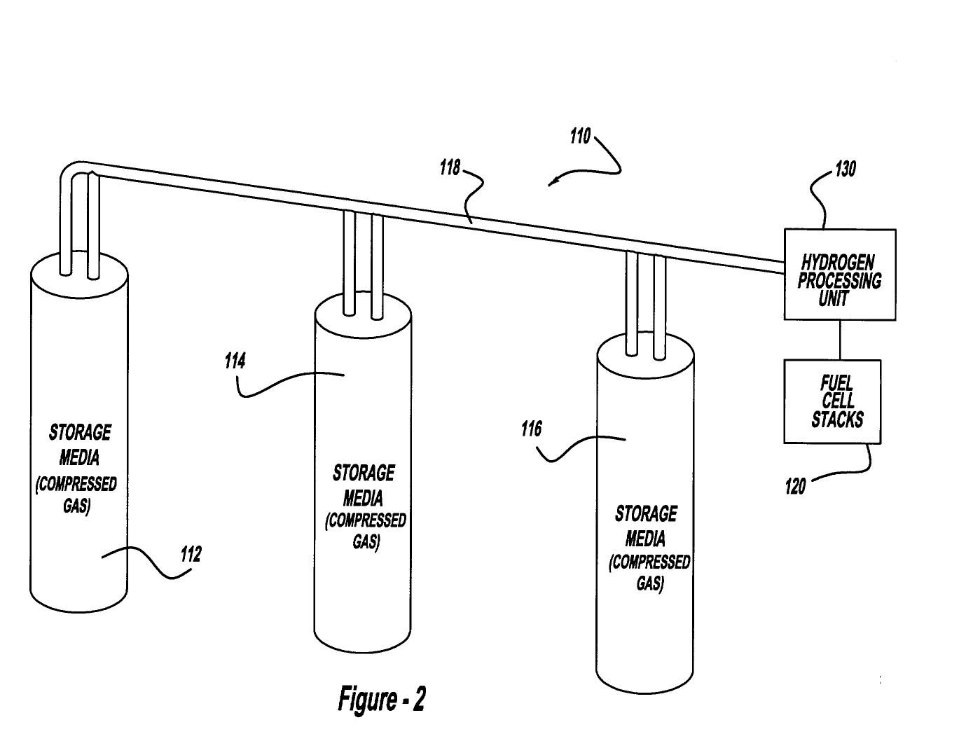

Turning to FIG. 2, a fuel cell system 110 is shown in accordance with the present invention to illustrate three compressed gas hydrogen storage tanks 112, 114, 116 connected to a common manifold 118 for delivery to a hydrogen processing unit 130 (configured as the hydrogen processing unit 30 described above) for delivering hydrogen at the desired pressure, temperature, humidity and purity to the fuel cell stacks 120.

While the best modes for carrying out the invention have been described in detail, those familiar with the art to which this invention relates will recognize various alternative designs and embodiments for practicing the invention within the scope of the appended claims.

1. A vehicle comprising:
a hydrogen processing unit connected to a fuel cell stack, wherein the fuel cell stack is configured to produce electric energy to power the vehicle; and
said hydrogen processing unit including a heat exchanger, a hydrogen filter, a hydrogen pressure regulator, and a hydrogen compressor to facilitate selective attachment of different types of hydrogen storage media to said hydrogen processing unit for delivering hydrogen gas to the fuel cell stack, the hydrogen storage media being selected from the group consisting of compressed gas, liquid, and solid hydride.

2. The vehicle of claim 1, wherein the hydrogen processing unit further includes a catalyst to enable attachment of hydrogen storage media in the form of chemical hydrides.

3. The vehicle of claim 1, wherein said hydrogen processing unit is configured to facilitate selective attachment of at least three different types of hydrogen storage media.

4. A hydrogen processing unit configured for attachment between a fuel cell stack and at least one hydrogen storage media on a fuel cell powered vehicle, the hydrogen processing unit comprising:
a heat exchanger;
a hydrogen filter;
a hydrogen pressure regulator; and
a hydrogen compressor;
said hydrogen processing unit being configured to receive hydrogen stored in any one of at least three different states including compressed gas hydrogen, liquid hydrogen, and solid hydride.

5. The hydrogen processing unit of claim 4, further comprising a catalyst in the hydrogen processing unit to enable attachment of hydrogen storage media in the form of chemical hydrides to the hydrogen processing unit.

6. The hydrogen processing unit of claim 4, wherein the hydrogen processing unit is configured to deliver hydrogen at a desired pressure, temperature, humidity and purity to the fuel cell stack.

7. The hydrogen processing unit of claim 4, wherein the hydrogen processing unit is configured to receive hydrogen gas from a solid hydrogen storage media stored in the form of sodium hydride or sodium borohydride.

8. A hydrogen processing unit configured for attachment between a fuel cell stack and at least one hydrogen storage media, the hydrogen processing unit comprising:
a heat exchanger;
a hydrogen filter;
a hydrogen pressure regulator;
a hydrogen compressor; and
a catalyst;
said hydrogen processing unit being configured to receive hydrogen stored in any one of at least three different states in the hydrogen storage media including compressed gas hydrogen, liquid hydrogen, and chemical or solid hydride, and said hydrogen processing unit being further configured to deliver hydrogen gas at a desired pressure, temperature, humidity and purity to the fuel cell stack.
